(19) United States
(12) Patent Application Publication
(10) Pub. No.: US 2018/0338743 A1
(43) Pub. Date: Nov. 29, 2018

(54) ACOUSTIC DETECTION MASK SYSTEMS AND/OR METHODS

(71) Applicant: ResMed Limited, Bella Vista (AU)

(72) Inventors: Liam HOLLEY, Sydney (AU); Robert John KING, Sydney (AU)

(21) Appl. No.: 16/053,723

(22) Filed: Aug. 2, 2018

Related U.S. Application Data
(63) Continuation of application No. 13/883,699, filed on May 6, 2013.
(60) Provisional application No. 61/344,894, filed on Nov. 5, 2010.

(51) Int. Cl. A61B 7/00; A61B 5/00; A61B 5/08

(52) U.S. Cl. CPC A61B 7/003; A61B 5/4818; A61B 2562/0204; A61B 5/682; A61B 5/6803; A61B 5/4806; A61B 5/08

(57) ABSTRACT

Certain examples described herein relate to acoustic detection mask systems and/or methods. In certain examples, an acoustic detection mask system is provided. An example acoustic detection mask system includes a mask having a microphone located therein or thereon. The microphone is connected to a data logger that is configured to capture vibrations and/or sounds registered by the microphone. The data logger may store such information in a computer readable storage media thereof for subsequent analysis, e.g., via a computer program accessing such data after the data logger is connected to a separate computer system. The microphone may be positioned and the data analyzed so as to determine differences between oral and nasal breathing, as well as sleep-disordered breath and/or snoring. Such components may be provided as a part of a system or in any suitable combination or sub-combination. Associated methods also are described herein as a part of the technology.

### CROSS REFERENCE TO APPLICATIONS

This application claims the benefit of U.S. Provisional Application No. 61/344,894 filed Nov. 5, 2010, of which is incorporated herein by reference in its entirety.

### FIELD OF TECHNOLOGY

The technology described herein relates to detection and/or diagnostic techniques for sleep-disordered breathing and/or the like. More particularly, the technology described herein relates to acoustic detection mask systems and/or methods.

### BACKGROUND OF TECHNOLOGY

Sleep-disordered breathing (SDB) encompasses a group of disorders where the breathing pattern or quality of ventilation is abnormal during sleep. Obstructive sleep apnea (OSA), is characterized by repetitive closing or collapse of the upper airway and partial or complete diminution of breathing.

OSA is often accompanied by snoring. Thus, many CPAP devices seek to determine the occurrence of snoring and to quantify it. For example, U.S. Pat. No. 6,840,907 provides a device having a sensor array and processor that is capable of analyzing snore. In particular, it measures the snore amplitude of the patient by passing digitized snoring signals through a high pass filter with a low frequency cut-off of approximately 10 Hz, calculating the modulus of each resulting signal, summing all the moduli, and passing the sum through a low pass filter with a high frequency cut-off of between 0.5 and 2 Hz. The processor also measures the harmonic purity of the patient's snore, that is, its closeness in form to a simple sine wave on the assumption that a non-obstructive snore has a different degree of harmonic purity than an obstructive one. Further, the system produces a measure of the harmonic stability of the patient's snore, that is, the accuracy with which one cycle of the snore signal matches its predecessor on the assumption that a non-obstructive snore has a different degree of harmonic stability than an obstructive one.

U.S. Pat. No. 6,705,315 describes a CPAP apparatus having a sound transducer and a system that responds to sound indicative of snoring.

For effective delivery of treatment pressure in an auto-titrating CPAP device, the treatment may be applied at the first sign of impending obstruction. In many patients, an apneic (obstructive) episode is associated with, e.g., preceded and/or followed, by a snore. Therefore, detection of snore is sometimes beneficial when attempting to preemptively delivery therapy.

U.S. Publication No. 2008/0308105, assigned to the assignee of the instant invention, describes a positive airway pressure (PAP) apparatus that determines the presence of a snore by the simplified method of using filtered expiratory noise as the measure of trinsic device noise and comparing that to filtered inspiratory noise. The filtering time constants for inspiratory and expiratory noise are adjusted such that treatment pressure has a reduced likelihood of causing false snore detection. In this way, economical, simplified techniques for detecting snoring in a patient receiving pressurized air from a PAP device are provided.

The entire contents of the above-listed patents and published patent application are hereby incorporated herein by reference.

However, the largest factor preventing treatment of sleep apnea and other forms of sleep-disordered breathing is believed to be the very low rates of diagnosis. Many current devices for detecting OSA, for instance, are hard for patients to access, as they are located in sleep labs and therefore require scheduled time away from the home, e.g., for participation in a sleep study. Other current devices for detecting OSA, for instance, are quite expensive. And still other current devices simply are not effective.

Thus, it will be appreciated that there is a need in the art for improved assessment techniques.

### SUMMARY OF TECHNOLOGY

An example of the present technology relates to a mask system, the mask system comprising a sensor or transducer adapted to detect noise from the patient's airways.

An example of the present technology relates to a mask system, the mask system comprising a sensor or transducer adapted to detect noise from the patient's airways, the microphone being so positioned to detect if the noise is from the patient's mouth or the patient's nose, or both.

An example of the present technology relates to a mask system, the mask system comprising a sensor or transducer, wherein the sensor or transducer is a microphone.

An example of the present technology relates to a mask system, the mask system comprising a sensor or transducer connected to a data logger.

An example of the present technology relates to a mask system, the mask system comprising a sensor or transducer, the mask having attachment regions proximal the patient's mouth.

An example of the present technology relates to a mask system, the mask system comprising a sensor or transducer, the mask having a malleable component adapted to shape the mask to the patient's face, including for example the patient's nose.

An example of the present technology relates to a mask system, the mask system comprising a sensor or transducer, the mask not forming a seal with the patient's airways.

In certain examples of the technology described herein, an acoustic detection mask system is provided. An example acoustic detection mask system includes a mask having a microphone located therein or thereon. The microphone is connected to a data logger that is configured to capture vibrations and/or sounds registered by the microphone. The data logger may store such information in a computer readable storage media thereof for subsequent analysis, e.g., via a computer program accessing such data after the data logger is connected to a separate computer system. The microphone may be positioned and the data analyzed so as to determine differences between oral and nasal breathing, as well as sleep-disordered breath and/or snoring. Such components may be provided is a part of a system or in any suitable combination or sub-combination. Associated methods also are described herein as a part of the technology.

Certain examples of the technology relate to an acoustic mask system and related techniques. The acoustic mask system of certain examples of the technology described herein involves a mask having a microphone connected thereto, as well as a data logger. The data logger records data from the patient, which data may be later analyzed to help diagnose the patient. The positioning of the microphone relative to the overall mask advantageously makes it possible to distinguish between different types of breathing, snoring, etc., and may allow the data to be processed so as to distinguish apneic, hypopneic, and/or other sleep-disordered breathing events. These example components will be described in greater detail below. Such components may be provided as a part of a system or in any suitable combination or sub-combination. Associated methods also are described herein as a part of the technology.

An example aspect of the disclosed technology relates to the overall acoustic mask system.

One example aspect of the disclosed technology relates to a mask for use with the overall acoustic mask system.

Another example aspect of the disclosed technology relates to mask designs that are adapted to form a seal with the patient's face and/or that are not adapted to seal with the patient's face.

Another example aspect of the disclosed technology relates to at least one area on the mask facilitating attachment of the mask to the patient's face. For instance, an adhesive area, e.g., first and second adhesive areas for attachment, e.g., to right and left sides of the patient's face, respectively. Other forms of the technology also are envisioned such as, for example, mechanisms coupling the mask to the head, e.g., via headgear supports that fit over the ears, eyes (such as glasses), around the patient's head etc.

Another example aspect of the disclosed technology relates to at least one strap of the mask adapted to fit at least partly around at least a portion of the patient's head for facilitating attachment of the mask to the patient's face.

Another example aspect of the disclosed technology relates to forming the mask from a flexible textile material. For instance, inner and outer components may be provided, with at least one being formed from a flexible textile material.

Another example aspect of the disclosed technology relates to at least one deformable element for fitting the mask to the patient's face. For instance, the at least one deformable element may be located in or on the mask and may be adapted to directly or indirectly contact a bridge or tip of the patient's nose. As another example, multiple deformable element may be provided, e.g., from for being proximate to the nose and/or chin.

Another example aspect of the disclosed technology relates to a microphone in or on the mask. The microphone may be, for instance, a contact/moving coil, condenser, dynamic, ribbon, PZT, or other type of microphone. Similarly, polar patterns such as, for example, omnidirectional, cardioid, boundary, etc., may be used in certain example instances.

Another example aspect of the disclosed technology relates to locating the microphone in or on the mask such that, when the mask is attached to the patient's face, the microphone is proximate to the patient's upper lip in a vertical direction and proximate the center of the patient's face in a horizontal direction.

Another example aspect of the disclosed technology relates to using the microphone of the mask to register noise/vibrations resulting from at least the patient's oral and nasal breathing, as well as the patient's snoring. For instance, the noise/vibrations registered may vary in dependence on the type of breathing, as well as the patient's snoring, such that the vibrations are distinguishable from one another based on the type of breathing, as well as the patient's snoring.

Another example aspect of the disclosed technology relates to using the microphone such that the sounds registered vary in dependence on the type of breathing, as well as the patient's snoring, and such that the sounds are distinguishable from one another based on the type of breathing, as well as the patient's snoring.

Still another example aspect of the disclosed technology relates to a data logger for use with the overall acoustic mask system.

Still another example aspect of the disclosed technology relates to a second microphone, in or on the data logger, which is configured to detect background, ambient, and/or environmental noise.

Still another example aspect of the disclosed technology relates to a computer connection supporting a data connection between the data logger and an external computer system.

Still another example aspect of the disclosed technology relates to a storage medium of the data logger that stores an analysis program executable by an external computer system after the data connection between the data logger and the external computer system is established.

Still another example aspect of the disclosed technology relates to an analysis program that is configured to automatically execute when the data connection between the data logger and an external computer system is established.

Still another example aspect of the disclosed technology relates to executing the analysis program directly from the data logger.

Yet another example aspect of the disclosed technology relates to a computer analysis program. For instance, the analysis program may comprise instructions, that when executed by at least one processor, causes the at least one processor to execute method steps.

Yet another example aspect of the disclosed technology relates to an informational card with which the data logger is packaged. For instance, the card may be foldable and may contain information regarding the use of the acoustic mask system or a component thereof (such as, for example, the mask, the data logger, the microphone, the computer analysis program, etc.), information as to what to do when the test cycle is completed, where to obtain treatment and/or further information, etc.

Yet another example aspect of the disclosed technology relates to a microphone for use with an acoustic mask system, e.g., as described herein. In certain example cases, the microphone may be configured to register sound, vibration, and/or noise due to patient nasal breathing, mouth breath, and/or snore with a sufficient level of detail or at a sufficient level of fidelity such that the sound, vibration, and/or noise can be classified as being caused by patient nasal breathing, mouth breath, and/or snore. Similarly, in certain examples of the technology disclosed herein, an analysis program configured to obtain data from the microphone, a data logger, a computer system, etc., may be controllable to distinguish sound, vibration, and/or noise due to patient nasal breathing, mouth breath, and/or snore.

A further example aspect of the disclosed technology relates to detecting a likely apneic and/or hypopneic events in registered sound.

A further example aspect of the disclosed technology relates to approximating a noise level by calculating an envelope of the microphone signal. For instance, the calculating of the envelope may be practiced by taking the square root of the sum of the signal squared and the Hilbert Transform of the signal.

A further example aspect of the disclosed technology relates to identifying a cession in flow by identifying a drop in the envelope below a first predefined threshold level. In certain instances, the first predefined threshold level may be adjusted based on background noise, stationary noise, and/or electrical interference. A likely apneic event may be taking or have taken place when the cession lasts for at least a first predefined amount of time.

A further example aspect of the disclosed technology relates to identifying at least one breathing cycle based on minima in the envelope.

A further example aspect of the disclosed technology relates to identifying a likely hypopneic event when the envelope indicates a reduction in flow below a second predefined threshold level for at least a second predefined amount of time.

A further example aspect of the disclosed technology, relates to improving the detecting of the likely apneic and/or hypopneic events by cross-referencing the detecting with signals from an oximeter, pressure or flow sensor, temperature sensor, and/or humidity sensor.

A further example aspect of the disclosed technology relates to calculating a probability for the detected likely apneic and/or hypopneic events.

A further example aspect of the disclosed technology relates to estimating total airway resistance and/or compliance at an airway resonant frequency.

A computer readable storage medium may comprise instructions that perform these and/or other aspects when executed by a computer.

According to certain examples of the technology disclosed herein, an acoustic mask system is provided. A mask is adapted to attach to a patient's face, with the mask comprising a microphone configured to register sound resulting from at least the patient's oral and nasal breathing, as well as the patient's snoring. A data logger is connected to the microphone of the mask, with the data logger including a non-transitory computer readable storage medium configured to store data representing the sound registered by the microphone.

According to certain examples of the technology disclosed herein, an acoustic mask system is provided. A mask is adapted to attach to a patient's face and is further adapted to vibrate in dependence on different types of airflows from the patient such that different types of breathing and/or snoring respectively generate identifiable and distinguishable vibrations in the mask, with the mask comprising a microphone configured to register sound resulting from any generated vibrations. A data logger connected to the microphone of the mask, with the data logger including a non-transitory computer readable storage medium configured to store data representing the sound registered by the microphone.

According to certain examples of the technology disclosed herein, an acoustic mask system is provided. A mask is adapted to attach to a patient's face, with the mask comprising a microphone configured to receive sound resulting from airflows generated by the patient, and with the sound being classifiable as one of a plurality of different types of breathing and/or snoring by virtue of the sound received by the microphone. A data logger is connected to the microphone of the mask, with the data logger including a non-transitory computer readable storage medium configured to store data representing the sound registered by the microphone.

According to certain examples of the technology disclosed herein, there is provided an acoustic mask for use with an acoustic mask system comprising the mask and a data logger configured to receive sound signals from a microphone of the acoustic mask. The mask is adapted to attach to a patient's face. The microphone of the mask is configured to register sound resulting from at least the patient's oral and nasal breathing, as well as the patient's snoring. The sound received by the microphone varies in dependence on the type of breathing, as well as the patient's snoring, such that sounds are distinguishable from one another based on the type of breathing, as well as the patient's snoring.

According to certain examples of the technology disclosed herein, there is provided a data logger for use with an acoustic mask system comprising a mask adapted to attach to a patient's face. The mask comprises a microphone configured to register sound resulting from at least the patient's oral and nasal breathing, as well as the patient's snoring. The data logger comprises: at least one processor; a microphone input configured to receive output from the microphone of the mask; and a non-transitory computer readable storage medium configured to store data representing the sound registered by the microphone.

According to certain examples of the technology disclosed herein, there is provided a non-transitory computer readable storage medium storing a program comprising instructions that, when executed by a processor, cause a computer to perform method steps. The method steps include, for example, accessing data representing sounds registered by a microphone of a mask in an acoustic mask system comprising the mask and a data logger, with the sound resulting from any oral and nasal breathing of a patient, as well as any snoring by the patient; and analyzing the data representing the sound registered by the microphone of the mask.

According to certain examples of the technology disclosed herein, there is provided a method of capturing data from a mask connected to an acoustic mask system that includes the mask, a microphone connected to the mask, and a data logger. The method comprises, for example, attaching the mask to a patient's face; connecting the microphone to the data logger; and storing data representing sound registered by the microphone on a storage medium of the data logger, with the sound being generated as a result of vibrations of the mask corresponding to at least the patient's oral and nasal breathing, as well as the patient's snoring, and with sounds being distinguishable from one another and each identifying a corresponding type of breathing and/or snoring.

According to certain examples of the technology disclosed herein, there is provided a method of analyzing data collected from a mask connected to an acoustic mask system that includes the mask, a microphone connected to the mask, and a data logger. The method comprises, for example, connecting the data logger to a computer via a computer connection; executing a program storing instructions that, when executed, cause the computer to analyze the data; and displaying results of said analyzing via the computer system, and/or transmitting the results to a remote location for further analysis. The data represents sounds registered by the microphone connected to the mask, with the sound being generated as a result of vibrations of the mask corresponding to at least the patient's oral and nasal breathing, as well as the patient's snoring, and with sounds being distinguishable from one another and each identifying a corresponding type of breathing and/or snoring.

According to certain examples of the technology disclosed herein, data may first be captured and then analyzed, e.g., in accordance with the examples described herein.

Other aspects, features, and advantages of this technology will become apparent from the following detailed description when taken in conjunction with the accompanying drawings, which are a part of this disclosure and which illustrate, by way of example, principles of this technology.

### BRIEF DESCRIPTION OF THE DRAWINGS

The accompanying drawings facilitate an understanding of the various examples of this technology. In such drawings:

FIG. 1A is a simplified view demonstrating a working principle for certain examples in which air diffusion from an oral flow through a mask creates detectable vibrations, sound, noise, and/or the like;

FIG. 1B is a simplified view demonstrating a working principle for certain examples in which air diffusion from a nasal flow through a mask creates detectable vibrations, sound, noise, and/or the like;

FIG. 1C is a simplified view demonstrating a working principle for certain example in which air diffusion through a mask resulting from snoring creates detectable vibrations, sound, noise, and/or the like;

FIG. 2 is a partial perspective view of an acoustic mask system in accordance with certain examples of the technology disclosed herein;

FIG. 3 is a partial perspective view of an acoustic mask in accordance with certain examples of the technology disclosed herein;

FIG. 4 is an exploded partial schematic view of an acoustic mask in accordance with certain examples of the technology disclosed herein;

FIG. 5 is an exploded partial schematic view of a data logger for use with an acoustic mask system in accordance with certain examples of the technology disclosed herein;

FIG. 6 is an enlarged partial perspective view of a data logger for use with an acoustic mask system in accordance with certain examples of the technology disclosed herein;

FIGS. 7A and 7B show example steps for attaching an example acoustic mask to a patient's face in accordance with certain examples of the technology disclosed herein;

FIG. 7C shows an example acoustic mask on a patient's face in accordance with certain examples of the technology disclosed herein;

FIG. 8 is a block diagram showing illustrative components of a data logger for use with an acoustic mask system in accordance with certain examples of the technology disclosed herein;

FIG. 9 is a flowchart showing illustrative steps for using the acoustic mask system of certain examples of the technology disclosed herein;

FIG. 10 is an example view showing an illustrative data logger connected to a computer system in accordance with certain examples of the technology disclosed herein;

FIG. 11 is a graph illustrating example sound pressures in the mask for “normal” breathing;

FIG. 12 is a graph showing an example microphone signal;

FIG. 13 is a graph showing an example of where the envelope has been calculated by taking the square root of the sum of the signal squared and the Hilbert Transform of the signal in accordance with certain examples of the technology disclosed herein;

FIG. 14 is a graph that in which a breath period, including expiration and inspiration periods, are identified, e.g., in connection with the detection of minima;

FIG. 15 is a graph that shows a likely apneic event; and

FIG. 16 is a graph that shows a likely hypopneic event.

### DETAILED DESCRIPTION OF ILLUSTRATED EXAMPLES

The following description is provided in relation to several examples (most of which are illustrated, some of which may not) which may share common characteristics and features. It is to be understood that one or more features of any one example may be combinable with one or more features of the other examples. In addition, any single feature or combination of features in any of the examples may constitute additional examples.

In this specification, the word “comprising” is to be understood in its “open” sense, that is, in the sense of “including”, and thus not limited to its “closed” sense, that is the sense of “consisting only of”. A corresponding meaning is to be attributed to the corresponding words “comprise”, “comprised”, and “comprises” where they appear.

The term “air” will be taken to include breathable gases, for example air with supplemental oxygen.

Certain examples of the technology disclosed herein relate to an acoustic mask system and related techniques. The acoustic mask system of certain examples of the technology disclosed herein involves a mask having a microphone connected thereto, as well as a data logger. The data logger records data from the patient, which data may be later analyzed to help diagnose the patient. The positioning of the microphone relative to the overall mask advantageously makes it possible to distinguish between different types of breathing, snoring, etc., and may allow the data to be processed so as to predict apneic, hypopneic, and/or other sleep-disordered breathing events. These example components will be described in greater detail below. Such components may be provided as a part of a system or in any suitable combination or sub-combination. Associated methods also are described herein as a part of the technology.

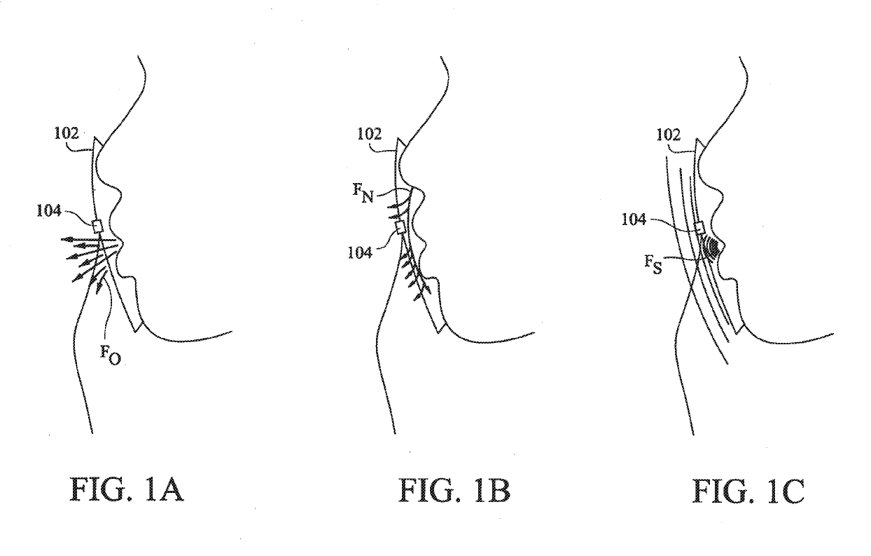

Referring now more particularly to the drawings in which like numerals indicate like components throughout the several views, FIGS. 1A-1C demonstrate working principles for certain examples of the technology disclosed herein. These figures collectively show a mask 102 having a sensor or transducer, such as a microphone, 104 connected or attached thereto. As the patient breaths in and out, the air flow vibrates within the mask and creates sounds. These sounds may be detected by the microphone 104. The inventors have discovered that the mask 102 can be made to vibrate and/or produce a sound if the microphone 104 is appropriately positioned relative to the overall mask 102. The inventors also have discovered that different types of breathing may produce unique, or at least distinguishable, vibrations and/or sounds. For instance, oral breathing will in such cases have a different sound than nose breathing. Various different example flows are shown schematically in FIGS. 1A-1C. It will be appreciated that certain examples of the technology disclosed herein may monitor the sound of the air from the patient and/or the sound that exhaled air makes against the mask material. In certain example, both may be measured (e.g., by respective microphones) and they may be compared, e.g., for cross-checking purposes or the like.

FIG. 1A is a simplified view demonstrating a working principle for certain examples in which air diffusion from an oral flow through a mask creates detectable vibrations, sound, noise, and/or the like. Air flow from the mouth (F_{O}) may flow substantially perpendicular to the vertical plane of the patient's face and also the vertical plane of the mask 102.

In contrast with FIG. 1A, FIG. 1B is a simplified view demonstrating a working principle for certain examples in which air diffusion from a nasal flow through a mask creates detectable vibrations, sound, noise, and/or the like. Air flow from the nose (F_{N}) may flow substantially parallel or have a parallel component to the vertical plane of the patient's face and the mask 104. Indeed, as can be seen from FIG. 1B, there is a significant substantially vertical component to the air flow from the nose F_{N}. Therefore, the sound created by the air flow from the mouth (e.g., as in FIG. 1A) may be different from the sound of the air flow from the nose (e.g., as in FIG. 1B).

It is useful to know if the patient habitually nose and/or mouth breaths as this information may be utilized by a physician or doctor in order to determine the patient's breathing habits, and if they are to prescribe CPAP treatment, which mask or mask system may be most appropriate for the patient (e.g. nasal, full face, nasal with a chin strap).

Still further, FIG. 1C is a simplified view demonstrating a working principle for certain examples in which air diffusion through a mask resulting from snoring creates detectable vibrations, sound, noise, and/or the like. As can be seen from FIG. 1C, snoring signals may be received and detected, and possibly ultimately distinguished from any detected oral and/or nasal flows, as they each may have a different sound pattern including distinct vibration patterns.

In certain examples of the technology disclosed herein, the microphone may be positioned such that substantially no sound is detectable by unobstructed oral and/or nasal breathing. This may be accomplished, for example, if the amount of vibrations caused by such breathing is kept at a low level (e.g., at or below a threshold sensitivity level of the microphone).

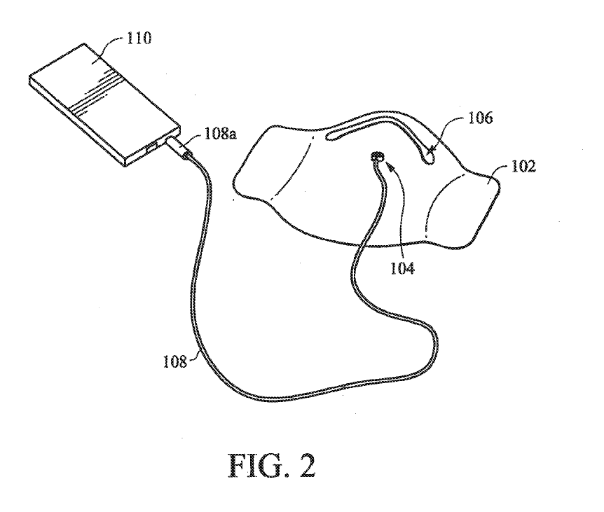

FIG. 2 is a partial perspective view of an acoustic mask system in accordance with certain examples of the technology disclosed herein. As indicated above, the acoustic mask system of certain examples includes a mask 102 that incorporates a microphone 104. At least one deformable element 106 may be included in the mask 102 so as to help it fit snugly and comfortably against the patient's face (e.g., the bridge of the nose). In the FIG. 2 example, the microphone 104 is in connected via a wire 108 with a data logger 110. In general, a patient wears the mask 102. The microphone 104 picks up sound caused, for example, by sounds and/or vibrations in the mask 102 that result from the mouth and/or nasal breathing, as well as snoring. Such data is recorded in the data logger 110 for later analysis, e.g., on a computer (not shown in FIG. 2). Further details on each of the components shown in FIG. 2 are provided below.

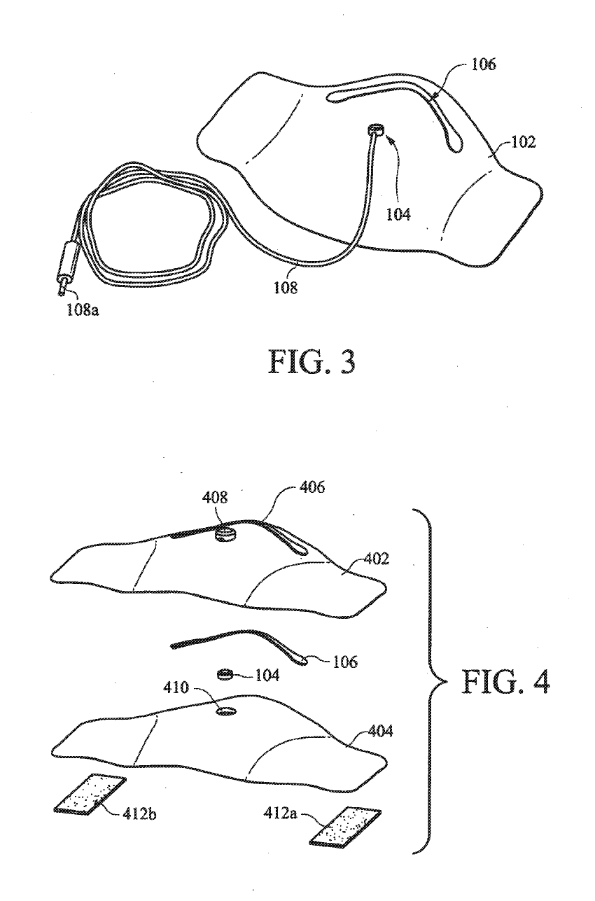

FIG. 3 is a partial perspective view of an acoustic mask 102 in accordance with certain examples of the technology disclosed herein. The mask 102 in general may be made from a fabric or soft component that may adhesively attach to the patient's face. It may also be attached by straps and/or other attachment means. The fabric may be polyester, cotton, felt, 3D webbing, elastic, weaves, or other suitable fabric. There may be a fabric on the patient contacting side and a second fabric on the non-patient contacting side. There also may be one or more internal layer(s) including other fabrics, spacers, foam, reinforcement, laminates, etc, or electronic equipment may be built in such as sensors, cables, microphone, etc.

The mask 102 may or may not seal. Although the mask 102 need not seal, in certain examples, the microphone 210 connected to the mask 102 should be able to register sounds from the patient's nose and/or mouth. In that regard, the mask 102 may cover both the nose and mouth of the patient in certain examples.

The mask 102 may be a “one-size-fits-all” type mask in certain examples, e.g., as in the case of the FIG. 3 example. However, in certain examples of the technology disclosed herein, different size masks may be provided (e.g., plural masks may be designated as small/medium/large/extra large, numbered, sized in inches or centimeters, etc.). In further examples, one-size-fits-all and/or differently sized masks may be at least partially adjustable, e.g., by virtue of straps, repositionable adhesive means, hook and loop, sliding, folding, overlapping, and/or suitable combinations thereof.

The mask 102 may be permeable (e.g., because it is formed from an air-permeable material) so as to aid in venting in certain examples. In addition, or in the alternative, one or more vents may be provided. In certain examples of the technology disclosed herein, the mask may not be permeable but have a distinct vent, whereas the mask may not seal to ensure venting from the mask in other examples.

As shown in FIG. 3, the mask 102 is attached to a cable 108 which, in turn, is connected to a data logger (not shown in FIG. 3 but described in greater detail below). The cable 108 may include a plug 108a at one end. The plug 108a may be, for example, an industry-standard plug such as a mini-plug, a micro-mini plug, and/or the like. In certain examples of the technology disclosed herein, the connection between the microphone 104 in the mask 102 and the data logger may be a fixed or removable wire connection. In certain of such cases, the cable 108 may or may not be removable from the microphone and/or data logger. In certain examples of the technology disclosed herein, a wireless connection may be provided between the microphone 104 in the mask 102 and the data logger.

FIG. 4 is an exploded partial schematic view of an acoustic mask in accordance with certain examples of the technology disclosed herein. The mask in the FIG. 4 example includes outer and inner portions 402 and 404. The outer portion 402 of the mask may have a feature 406 for accommodating a fitting element 106. In certain examples, the feature 406 may be, for example, a sleeve, pocket, or other compartment far holding the fitting element 106 in place. In certain other examples, the feature 406 may be a recess or hole for accommodating the fitting element 106. In certain examples, the fitting element 106 may be made from an at least partially deformable material such as, for example, aluminum. The fitting element 106 may provide at least initial shape to the overall mask. Depending on the malleability and/or overall deformability of the fitting element 106, a patient may be able to further customize the fit of the overall mask, e.g., by placing it over the nose and mouth, and squeezing the fitting element 106 into a comfortable place. Of course, other materials aside from, or in addition to aluminum may be used for fitting element 106. Such materials may include metals and metal alloys, plastics, polymers, and/or the like.

The fitting element 106 may be a rigid or semi-rigid component in certain examples of the technology disclosed herein. It may be placed aver at the nose bridge and/or at other areas to aid in positioning and/or sealing the mask. The fitting element 106 may be placed between layer(s) or fabric or other materials, and it may be mechanically or chemically attached to the mask (or a portion thereof such as, far example an inner and/or outer portion thereof). For instance, a rigid or semi-rigid element may be wholly enclosed by the layers by, for example, gluing, stitching, welding, ultrasonic welding, thermoforming, etc. The fitting element 106 may not be wholly within the layers in that, for example, a rigid or semi-rigid strip-like or other shaped element may have one or more exposed portion(s) to, far instance, provide a grip tab, enabling the patient to peel off the mask after a sleep session.

One or more fitting element(s) may be provided. For instance, two fitting strips may be provided for the nose bridge and just over the chin. Certain examples of the technology disclosed herein may use other shapes in place of or in addition to generally elongate strips. For example, a generally circular or ovular semi-rigid or rigid element may be provided so as to be positionable over both the nose bridge and above the chin, etc.

One or more sensors or transducers, for example a microphone, 104 may be provided to the mask, e.g., one tuned and/or positioned for each type of noise. For instance, a protrusion 408 in the outer portion 402 and/or of the mask and a recess or hole 410 (e.g., through-hole) in the inner portion 404 of the mask may be provided for accommodating the microphone 104. The protrusion 408 in the FIG. 4 example includes a small through-hole such that a cable 108 can be nm from the microphone to the data logger (e.g., as described in greater detail below). In certain examples of the technology disclosed herein, the microphone 104 may be provided in a fixed position, e.g., if it is sewn or otherwise integrated into the overall mask. However, in certain examples, the microphone 104 may be movable and/or repositionable, e.g., by the patient. For instance, the microphone 104 may be movable so that a patient can position it relative to a prescribed position on the mask. Such a position may be, for example, level with or slightly above the upper lip in substantially the center of the face in certain example instances.

In the FIG. 4 example, an adhesive material (not shown) is provided to a side of the inner portion 404 that is to contact the person's face. To at least initially protect the adhesive material, adhesive backings 412 may be provided. In the FIG. 4 example in particular, left and right adhesive backings 412a and 412b are provided, e.g., such that the mask can be adhered to the patients face at at least these positions. More or fewer adhesive locations and thus more or fewer adhesive backings 412 may be provided in different examples of the technology disclosed herein.

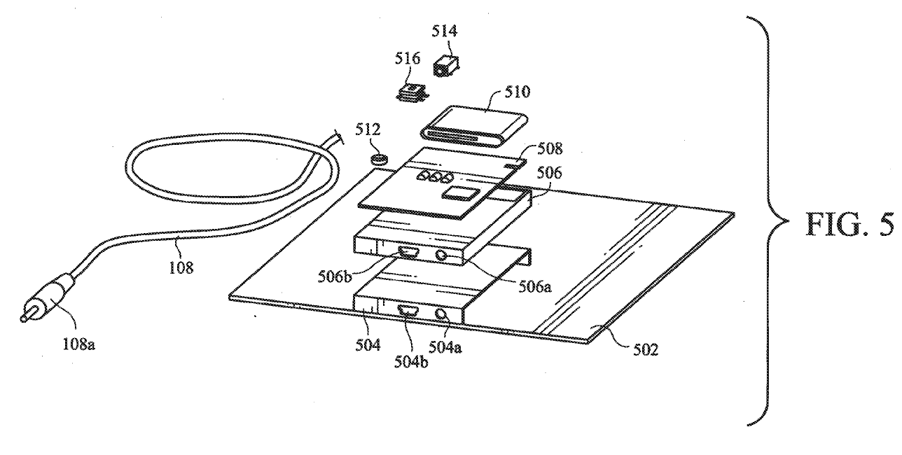

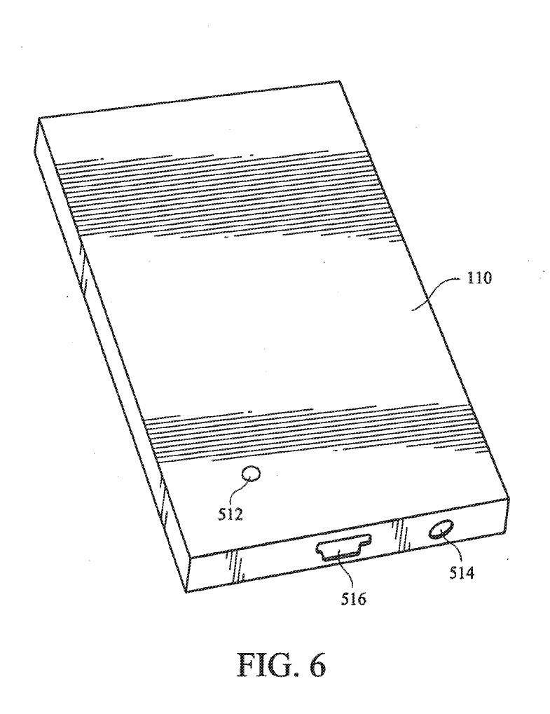

FIG. 5 is an exploded partial schematic view of a data logger 110 for use with an acoustic mask system in accordance with certain examples of the technology disclosed herein, and FIG. 6 is an enlarged partial perspective view of a data logger 110 for use with an acoustic mask system in accordance with certain examples of the technology disclosed herein. The data logger 110 a simple audio recording device containing storage medium 510 (e.g., a flash memory), a battery, and a printed circuit board (PCB) 508, logic card, or the like, that performs audio encoding. The data logger 110 in certain examples may be activated automatically, e.g., when the audio cable 108 is plugged in. This may be accomplished through the use of a pair of contacts that are connected by the plug 514 itself.

The data logger 110 may be packaged with printed material 502, e.g., on a card or the like. The printed material 502 may include instructions for use of the mask system and/or components thereof, contact information for a sleep clinic in the area from which the data logger 110 was acquired (e.g., purchased, borrowed, etc.), etc.

To help protect the components of the data logger, a first outer housing 504 may be provided. The first outer housing 504 may include openings 504a and 504b for accommodating plugs for the microphone on the mask and a computer (e.g., USB) connection. An inner housing 506 may have similar openings 506a and 506b, and may store the PCB 508, together with the storage medium 510, a microphone 512, and the microphone and computer connections 514 and 516. Although the storage medium 510 may be a flash memory (e.g., an EEPROM) in certain examples, other forms of non-transitory computer-readable storage media may be used in place of, or in addition to, the flash memory.

The microphone 512 on the data logger 110 may help capture background or ambient or environmental noise, e.g., so that the sounds captured by the microphone in the mask can be more adequately distinguished from one another and/or from such background or ambient or environmental noise. In certain examples, a hole may be formed in the inner and/or outer housings 504 and 506 so as to enable the microphone 512 to be operable therethrough. In certain cases, one or more additional microphones for detecting background or ambient or environmental noise may be provided to the mask rather than, or in addition to, the data logger 110.

A battery (not shown) also may be located within the inner housing 506 in certain examples and, in certain example instances, the battery may be rechargeable (e.g., by using the USB connection 516, through a dedicated power connection, etc.).

Although USB was mentioned as being suitable for the computer connection 516 was described above, other forms of computer connections may be provided in different examples of the technology disclosed herein. For instance, parallel, serial, USB, micro-USB, “firewire,” network, and/or other connections may be provided. In certain examples of the technology disclosed herein, when the data logger 110 is connected to a computer via the computer connection 516, the user may be given the option of running an application that performs signal analysis, displays results of the signal analysis, and/or transmits such results to a sleep clinician or the like. Such a computer program may run automatically, for instance, when a USB cable is connected to the computer. The data logger 110 in this context may essentially act as a removable storage location (e.g., to a Windows-compatible computer). The program may be stored on and/or executable directly or indirectly from the storage medium 510 in the data logger 110 in certain examples. In certain other examples, the program may be a more stand-alone program that is packaged and/or distributed together with or separate from the printed matter 502, downloadable from the Internet, etc. In certain examples, the program may be retrieved from the storage medium 510 of the data logger 110 the first time it is operably connected to a computer. Subsequently, the program may be removed from the storage medium 510 of the data logger 110, e.g., advantageously freeing up space thereon. In any event, the program may be stored on a computer readable storage medium and may include instructions that, when executed, perform corresponding method steps.

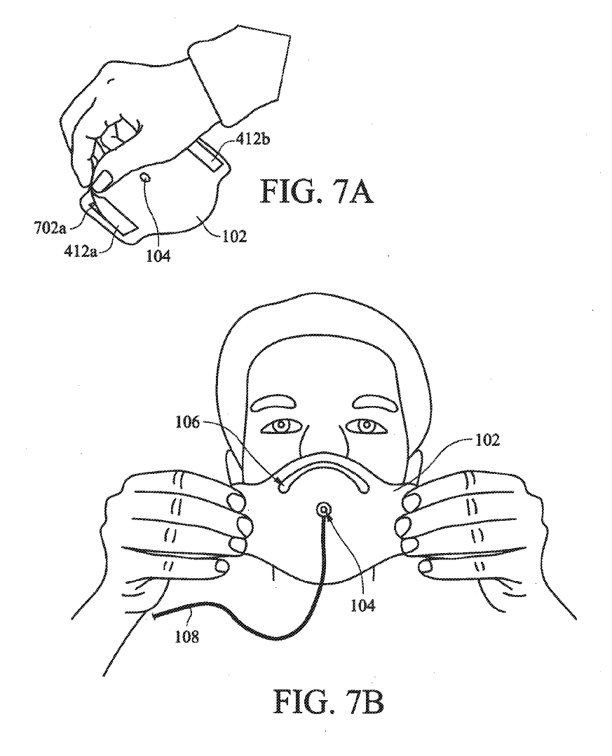

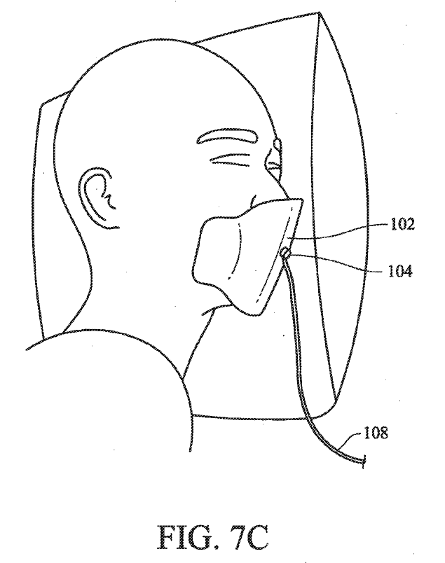

FIGS. 7A-7C illustrate how an example acoustic mask can be put on and worn in certain example instances. More particularly, FIGS. 7A and 7B show example steps for attaching an example acoustic mask to a patient's face in accordance with certain examples of the technology disclosed herein. The example mask in FIG. 7A has adhesive materials on its left and right sides. A patient can peel back or otherwise remove tabs covering from adhesive backings 412a and 412b so as to expose adhesive areas 702. The mask can be fit to the face as shown in FIG. 7B, for example. In certain examples, the deformable element 106 is located at the bridge of the wearer's nose, and the microphone 104 is vertically positioned at or just about the upper lip and horizontally in substantially the center of the wearer's face. In certain examples, the top of the mask can be aligned over the tip of the nose, e.g., along the bridge. Once initially positioned, as indicated above, the deformable element 106 may be squeezed or otherwise manipulated to ensure a good fit. FIG. 7C shows an example acoustic mask on a patient's face in accordance with certain examples of the technology disclosed herein.

As indicated above, other attachment means can be used in place of, or in addition to, the adhesive attachment means described in FIGS. 7A-7C. Straps and the like may be provided in certain examples, e.g., to provide a more snug and potentially secure fit. Straps also may be advantageous for patients with facial hair, sensitive skin, and/or the like.

The example mask shown in FIGS. 7A-7C is a non-sealing mask. However, as indicated above, other examples may involve sealing masks. Although the techniques of certain examples of the technology disclosed herein result in low-cost acoustic mask systems, certain examples may employ the techniques described herein, e.g., in connection with more expensive masks and/or flow generators. For instance, a patient thinking of a particular mask for a protracted treatment may “experiment” with it and, for example, at the same time also take advantage of the acoustic measurement techniques herein. In operation, the microphone may be positioned so as to reduce the amount of vibration and/or sound caused from the supply of therapeutic pressurized gas being provided. In addition, or in the alternative, the processing program may be configured to reduce or eliminate vibrations and/or sounds generated by the therapeutic air flow, the flow generator, etc. In certain examples of the technology disclosed herein, the data logger 110 may be provided separate from, or together with, a controllable flow generator.

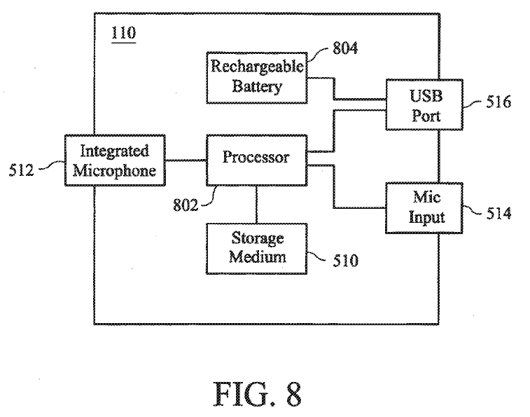

FIG. 8 is a block diagram showing illustrative components of a data logger 110 for use with an acoustic mask system in accordance with certain examples of the technology disclosed herein. As can be seen from FIG. 8, the data logger 110 includes at least one processor 802. The at least one processor is connected to the integrated microphone 512, as well as the microphone input 510. The processor 802 accepts input from the microphones connected to the data logger 110, and stores the data in the integrated storage medium 510 (which may include one or more storage locations). Suitable contacts may be provided on or proximate to the microphone input 510, e.g., to indicate that the data logger is to be turned on and to trigger the storage of data. The storage medium 510 itself may be removable from the data logger 110 in certain example.

A battery 804 (e.g., a rechargeable battery) may also be provided to the data logger 110 in certain examples. The battery 804 may be removable and/or replaceable in certain examples of the technology disclosed herein. In certain examples, the battery 804 may be rechargeable when powered from an external source, when the data logger 110 is connected to a computer system or other charge source via a computer connection (e.g., USB) port 516, etc.

The computer connection 516 may cause a program stored on the storage medium 510 to automatically execute when connected to the computer. This may cause, for example, the installation of a data analysis program on the computer, the running of a data analysis program directly from the data logger 110, etc. Further example functionality of such a computer program is described below.

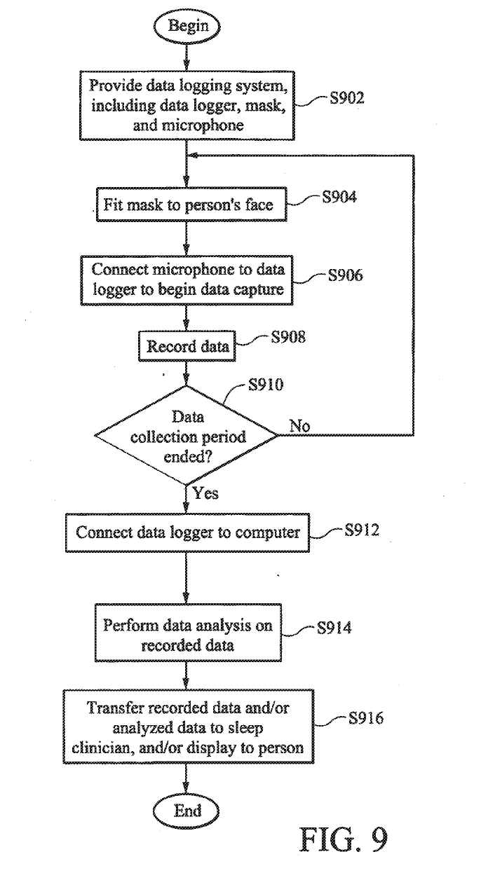

FIG. 9 is a flowchart showing illustrative steps for using the acoustic mask system of certain examples of the technology disclosed herein. A data logging system is provided in step S902. The data logging system of certain examples includes a data logger, a mask, and at least one microphone provided to the mask. The mask is fitted to or placed on the person's face in step S904. The microphone is connected to the data logger in step S906, which may begin the data capture in certain examples. In certain other examples, however, the data may not begin until a suitable action is taken (e.g., a start button or the like is pushed) and/or a suitable time has passed (e.g., such that the person is more likely to be asleep, a suitable amount of data concerning background noise is gathered, an “awake baseline” is obtained, etc). Once suitably connected, in step S908, data is recorded. Data may be collected for one or more sleep sessions. In step S910, a decision is made as to whether the data collection process has ended. If it has not (e.g., if the person is to wear the mask for one or more additional periods), then the process returns to step S904.

If, however, the data collection process is complete, the data logger may be connected to the computer in step S912. Data may, for example, be transmitted to the computer, which may have more or better resources for analyzing it. In any event, in step S914, data analysis is performed on the recorded data. This may take place on locally on the data logger, on the computer system to which the data logger is connected (e.g., based on data transferred to the computer system and/or left on the data logger), on a network location, etc. Recorded and/or analyzed data may be transferred to another location, e.g., to a sleep clinician, and/or displayed to the person locally via the computer system.

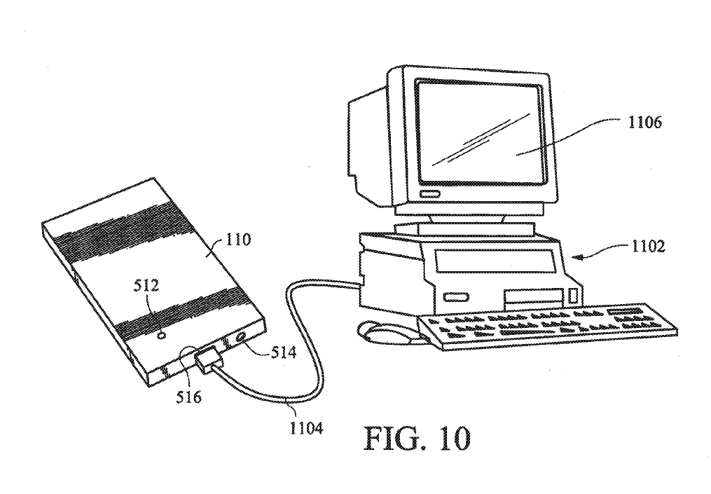

FIG. 10 is an example view showing an illustrative data logger connected to a computer system in accordance with certain examples of the technology disclosed herein. In FIG. 10, a data logger 110 is connected to a computer system 1102, e.g., via a cable 1104. As alluded to above, the cable may be a USA cable in certain examples. Once connected, the user may have direct or indirect access to the recorded data. For instance, a program executable via the computer system 1102 may provide a user interface that enables a user (who may in certain example instances be the patient, a sleep clinician, or other person) to at least visualize the data or a processed form thereof, e.g., on a display 1106 of the computer system 1102.

In certain examples of the technology disclosed herein, the program may analyze the data. For instance, the program may remove background or ambient or environmental noise (e.g., by contrasting the data from the mask microphone 102 with the data logger integrated microphone 512, etc.), as well as any other unexpected noises that may occur in use. This may be accomplished, for example, by use a suitable band pass or other filter on the data, compensating for the sounds detected by the data logger integrated microphone 512 (e.g., through simple subtraction, pattern matching techniques that attempt to associate loud sounds from the mask microphone 102 with corresponding sounds from the data logger integrated microphone 512, etc.), etc. Once a “clean” set of sounds is determined, further analysis may be performed, e.g., to classify the breathing as mouth or nasal breath, to detect snore, etc. Certain examples may plot or display such data, e.g., as frequency or event versus time. Summary statistics regarding number, timing, frequency, etc., of events also may be provided. Reasoning inferences regarding the presence of an abnormal event (e.g., an apneic and/or hypopneic episode) may be drawn, e.g., from long periods of no sound detection, etc. Of course, other data analysis may be performed by the program (or by the data logger 110), and other forms of data display, visualization, etc., are of course possible in different examples.

In certain examples of the technology disclosed herein, the data logger 110 may act as a “pass through” device. In certain examples, the data logger 110 may be connected to the computer system 1102 while it is gather data from the patient. In such cases, for example, the data logger 110 may simply pass registered sound and/or vibration data to the computer system 1102. In such cases, for example, the data logger 110 may pass along raw data for subsequent analysis, e.g., on the computer system 1102 or at a remote location, or it may process the data in some ways (e.g., by applying at least initial filtering and/or correlation techniques, etc.). The data in raw and/or processed form may be displayed on the display 1106 in real time or substantially real time in certain examples of the technology disclosed herein.

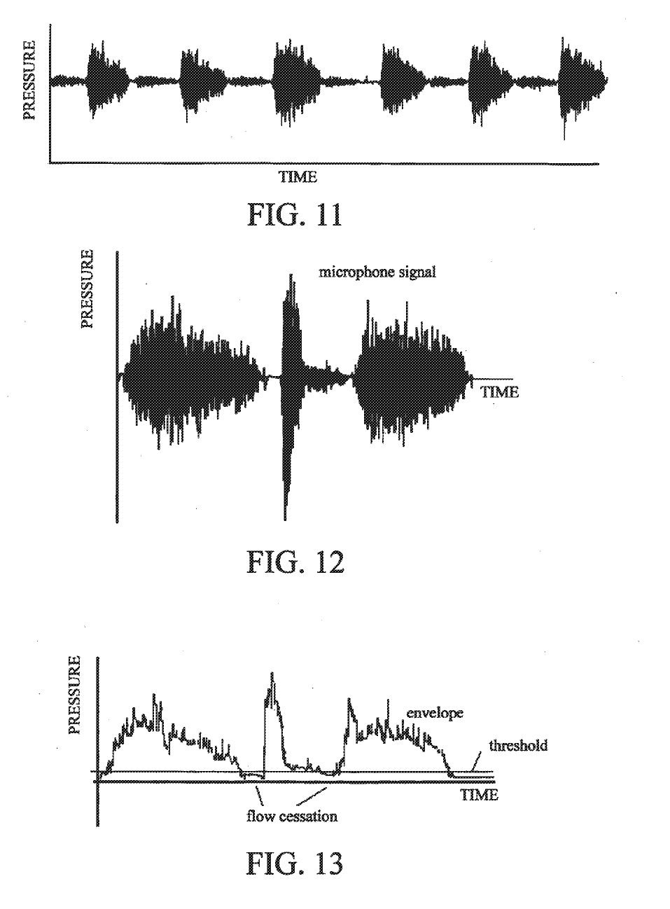

Example diagnostic and detection techniques usable in connection with certain examples of the technology described herein will now be provided. Breathing produces turbulent flow in the airways, and potentially in the mask. Noise level has been found to typically increase with turbulence. FIG. 11 is a graph illustrating example sound pressures in the mask for “normal” breathing.

The noise level may in certain cases be approximated by the envelope of the microphone signal. One technique for calculating the envelope involves taking the square root of the sum of the signal squared and the Hilbert Transform of the signal. The Hilbert Transform of the signal typically is 90 degrees (or about 90 degrees) out of phase with the signal, so it can be seen that at any point the signal magnitude is given by the hypotenuse between the signal and its Hilbert Transform. See, for example, “Practical use of the ‘Hilbert transform”’ by N. Thrane, J. Wismer, H. Konstantin-Hansen & S. Gade, Brüel & Kjær, Denmark, the entire contents of which are hereby incorporated herein by reference. Of course, it will be appreciated that other techniques of determining the envelope may be used in different examples.

Cessation of flow may be detected as corresponding to a drop in the envelope below a threshold level. The threshold may be automatically adjusted, e.g., to account for background noise, stationary noise, or the like including, for example, electrical interference. FIG. 12 is a graph showing an example microphone signal, and FIG. 13 is a graph showing an example of where the envelope has been calculated by taking the square root of the sum of the signal squared and the Hilbert Transform of the signal in accordance with certain examples of the technology disclosed herein. The flow cession period, as well as the threshold can be seen in the FIG. 13 example.

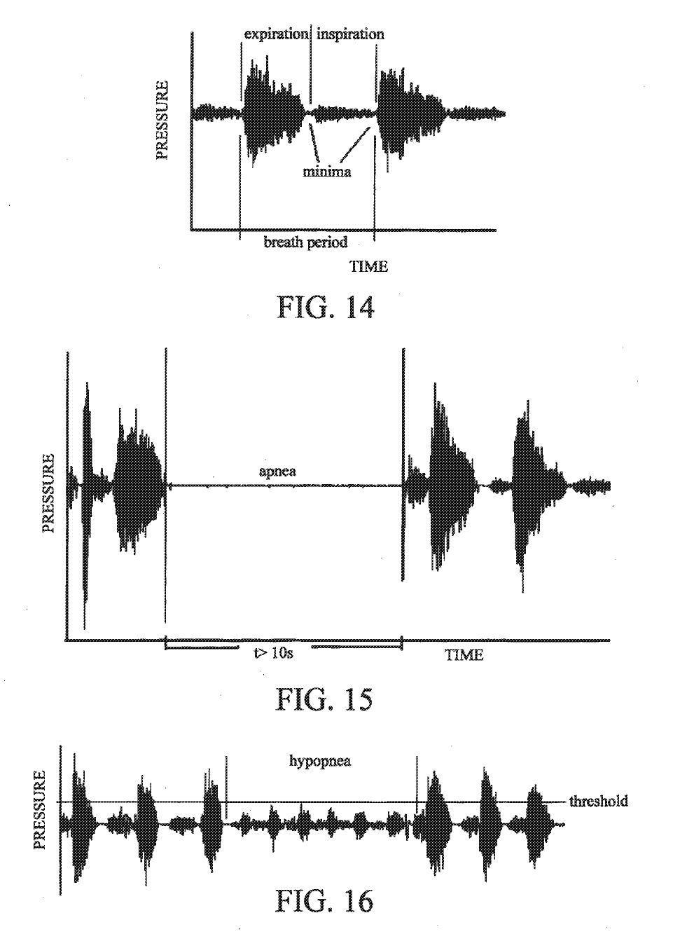

Between inspiration and expiration, the flow in the airway momentarily stops. Thus, the noise produced by turbulent airflow will cease, and the sound level recorded by the microphone will be reduced. Breathing can be detected as periods of turbulent noise with minima occurring at twice the respiratory rate, for example, where the respiratory rate may be assumed to occur within a range of values. A probability of apnea may be indicated by a lack of detection of breathing for a defined period, e.g., 10 seconds. If a cessation of flow lasts longer than a particular length of time it may be associated with an apnea. For example, if flow ceases for more than 10 seconds it may be an indication of apnea, or as an indication of a particular probability of an apnea. See, for example, FIG. 14, which is a graph that in which a breath period, including expiration and inspiration periods, are identified, e.g., in connection with the detection of minima.

In certain examples of the technology disclosed herein, a microphone may be used in conjunction with an oximeter, e.g., such that when the oximeter shows a particular oxygen desaturation in conjunction with a prolonged drop in the sound pressure envelop below a threshold, these indications may be used to suggest a particular probability of apnea. In further examples, the acoustic mask system may be used in conjunction with other sensing techniques including, for example, pressure or flow, temperature, humidity sensing, etc. Joint probabilities of various events might be analyzed to indicate apnea in certain examples. FIG. 15 is a graph that shows a likely apneic event.

In addition to the example apnea detection/diagnostic techniques described above, certain examples may in addition or in the alternative involve example hypopnea detection. Hypopnea generally occurs when there is a reduction in flow for a prolonged period, for example, 10 seconds. If hypopnea is caused by reduced central drive, it may be detected as a drop in the peak of the envelope of the microphone signal, e.g., within a series of breaths below a particular threshold value.

If hypopnea is caused by increased airway resistance, it may also accompany increased turbulence and an increase in the noise level. This can be accompanied by more chaotic and impulsive features in the microphone signals. There may also be more harmonic components in the sound, particularly appearing within frequency ranges also associated with snore noises. Joint probabilities of these features and additional features can be used to determine the probability of hypopnea and other events based on prior knowledge of the probability of such events and their associated features.

FIG. 16 is a graph that shows a likely hypopneic event. As can be seen, the somewhat more chaotic and/or harmonic features of the likely hypopneic event occur below the threshold selected for the FIG. 16 example.

It will be appreciated that certain examples of the technology disclosed herein may be capable of estimating the total airway resistance and compliance at an airway resonant frequency. The frequency of the resonance can be determined by the highest peak in the spectrum, averaged spectrum within a particular frequency range, etc. The total airway resistance at this frequency has been found to be proportional to the width of the peak 3 dB below its maximum in certain example cases. The ratio of the oscillating mass of air in the airway to the total stiffness of the airway also has been found to be proportional to the frequency of the peak squared in certain example cases. Using these example techniques, for instance, it is possible to infer characteristics regarding overall airway mechanics.

While the technology has been described in connection with several examples, it is to be understood that the technology is not to be limited to the disclosed examples, but on the contrary, is intended to cover various modifications and equivalent arrangements included within the spirit and scope of the technology. Also, the various examples described above may be implemented in conjunction with other examples, e.g., one or more aspects of one example may be combined with aspects of another example to realize yet other examples. Further, each independent feature or component of any given assembly may constitute an additional example. In addition, while the technology has particular application to patients who suffer from OSA, it is to be appreciated that patients who suffer from other illnesses (e.g., congestive heart failure, diabetes, morbid obesity, stroke, bariatric surgery, etc.) can derive benefit from the above teachings. Moreover, the above teachings have applicability with patients and non-patients alike in non-medical applications.

1-85. (canceled)

85. A method of analyzing data collected from a mask connected to a mask system that includes the mask, a microphone connected to the mask, and a data logger, the method comprising:
executing a program storing instructions that, when executed, cause the computer to analyze the data; and
displaying results of said analyzing via the computer system, and/or transmitting the results to a remote location for further analysis,
wherein the data represents sound registered by the microphone connected to the mask, the sound being generated as a result of vibrations of the mask corresponding to at least the patient's oral and nasal breathing, as well as the patient's snoring, sounds being distinguishable from one another and each identifying a corresponding type of breathing and/or snoring.

86. The method of claim 85, further comprising detecting a likely apneic and/or hypopneic events in registered sound.

87. The method of claim 85, further comprising approximating a noise level by calculating an envelope of the microphone signal.

88. The method of claim 87, wherein the calculating of the envelope is practiced by taking the square root of the sum of the signal squared and the Hilbert Transform of the signal.

89. The method of claim 87, further comprising identifying a cessation in flow by identifying a drop in the envelope below a first predefined threshold level.

90. The method of claim 89, wherein the first predefined threshold level is adjusted based on background noise, stationary noise, and/or electrical interference.

91. The method of claim 87, further comprising identifying at least one breathing cycle based on minima in the envelope.

92. The method of claim 89, further comprising identifying a likely apneic event when the cession lasts for at least a first predefined amount of time.

93. The method of claim 87, further comprising identifying a likely hypopneic event when the envelope indicates a reduction in flow below a second predefined threshold level for at least a second predefined amount of time.

94. The method of claim 86, further comprising improving the detecting of the likely apneic and/or hypopneic events by cross-referencing the detecting with signals from an oximeter, pressure or flow sensor, temperature sensor, and/or humidity sensor.

95. The method of claim 86, further comprising calculating a probability for the detected likely apneic and/or hypopneic events.

96. The method of claim 86, further comprising estimating total airway resistance and/or compliance at an airway resonant frequency.

97-99. (canceled)
